(19) United States
(12) Patent Application Publication
(10) Pub. No.: US 2023/0355492 A1
(43) Pub. Date: Nov. 9, 2023

(54) Oral Care Compositions and Preservative Systems

(71) Applicant: Colgate-Palmolive Company, NEW YORK, NY (US)

(72) Inventors: Sandra PEREIRA RAMOS, Sao Paulo (BR); Fernanda CORREA, Sao Paulo (BR); Tatiana CINQUETTI, Sao Paulo (BR); Ariel HASKEL, East Brunswick, NJ (US); Natalia CASTRO DE SOUZA, Sao Paulo (BR); Jeffrey Merl MILLER, Jackson, NJ (US)

(73) Assignee: Colgate-Palmolive Company, New York, NY (US)

(21) Appl. No.: 17/632,176

(22) PCT Filed: Nov. 9, 2021
(86) PCT No.: PCT/US2021/058546

§ 371 Date: Feb. 1, 2022

Related U.S. Application Data
(60) Provisional application No. 63/111,379, filed on Nov. 9, 2020.

(51) Int. Cl. A61K 8/41; A61K 8/34; A61K 8/21; A61Q 11/00

(52) U.S. Cl. CPC A61K 8/416; A61K 8/34; A61K 8/21; A61Q 11/00; A61K 2800/524

(57) ABSTRACT

This invention provides, amongst other things, a preservative system for aqueous oral care compositions that comprises the addition of one or more quaternary ammonium antimicrobial agents (e.g., cetylpyridinium chloride (CPC)) in combination with comprising benzyl alcohol in a preservative system, can surprisingly allow for the use of relatively less benzyl alcohol while still maintaining antimicrobial efficacy and not adversely affecting the flavor of the composition. The added benefit in using relatively less benzyl alcohol, in addition to lower cost and expense, is also that the flavor or the overall composition is less impacted.

### BACKGROUND

This application relates, inter alia, to novel aqueous oral care compositions with improved preservative systems.

Various antibacterial agents can retard the growth of bacteria and other microbial agents, and thus serve as adequate antimicrobial preservative systems. In many cases, aromatic alcohols, for example, benzyl alcohol can be used. In other cases, there is a need to use other conventional or synthetic preservatives.

However, recently there has been various market pressures to more sustainable or “natural” materials. This has meant a push to preservative that are, for example, free of parabens, free of formaldehyde donors, halogenated preservatives, and isothiazolines. In short, developing preservatives and preservative combinations has become increasingly challenging. In turn, protection or preservation of aqueous or higher water formulations has been one area that has been especially challenging. Not all combinations of preservatives are necessarily compatible or effective together. This may be especially so when selecting preservative systems that incorporate ingredients that are consider “natural” rather than synthetic”. And no single preservative is necessarily equally effective against all types of microorganisms.

Further complicating matters, when a preservative system is not effective, there is the possibility for various physical changes to the product, which can include, but are not limited to: changes in pH, viscosity, color and odor. Ineffective preservative systems can also fail to hinder stability and separation of various ingredients, as well as degradation of active ingredients. Perhaps most concerning is the presence of obvious visible microbial growth. Other complications include the potential for a preservative to detrimentally effect flavor. For example, certain amounts of benzyl alcohol may have adverse impacts on flavor.

There is thus a need for novel oral compositions with preservative systems that are effective in protecting aqueous or higher water oral care formulations, but that do adversely affect flavor and are simultaneously cost-effective.

### BRIEF SUMMARY

In one embodiment, the addition of one or more quaternary ammonium antimicrobial agents (e.g., cetylpyridinium chloride (CPC)) in a preservative system comprising benzyl alcohol, can surprisingly allow for the use of relatively less benzyl alcohol—i.e., compared to other market formulations—while still maintaining antimicrobial efficacy and not adversely affecting the flavor of the composition. The added benefit in using less benzyl alcohol, in addition to lower cost and expense, is also that the flavor or the overall composition is less impacted.

The disclosure provides, in one embodiment, an aqueous oral care composition comprising:
(i.) a preservative system, wherein the preservative system comprises:
   a. an effective amount of benzyl alcohol; and
   b. an effective amount of one or more of quaternary ammonium surfactants (such as cetylpyridinium chloride (CPC));
   c. optionally a polysorbate.
(ii.) water

The disclosure further provides methods of inhibiting biofilm formation comprising administering to the oral cavity a composition as described.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In a first embodiment, the disclosure provides an aqueous oral care composition (Composition 1.0) comprising:
(i.) a preservative system, wherein the preservative system comprises:
   a. an effective amount of benzyl alcohol; and
   b. an effective amount of one or more of quaternary ammonium surfactants (such as cetylpyridinium chloride (CPC));
   c. optionally an effective amount of a polysorbate; and
(ii.) water.

For example, the disclosure provides embodiments of Composition 1.0 as follows (all amounts are by weight of the total composition):
1.1 Composition 1.0, wherein the one or more of quaternary ammonium surfactants is a pyridinium surfactant, e.g., cetylpyridinium chloride (CPC).
1.2 Composition 1.1, wherein the pyridinium surfactant is cetylpyridinium chloride (CPC) (e.g., in an amount from 0.005 to 0.05% by wt., relative to the total composition) (e.g., in an amount from 0.005 to 0.02% by wt.) (e.g., about 0.01% by wt.), (e.g., about 0.015% by wt.), (e.g., about 0.02% by wt.).
1.3 Any of the forgoing oral care compositions, wherein the preservative system further comprises an amino acid (e.g., a basic amino acid) (e.g., arginine).
1.4 Any of the forgoing oral care compositions, wherein the preservative system further comprises zinc ions.
1.5 Any of the foregoing oral care compositions, wherein the composition further comprises an orally acceptable salt selected from zinc salts, stannous salts, and bisguanide salts.
1.6 Any of the foregoing oral care compositions, wherein the composition further comprises metal ions supplied by one or more of stannous chloride, stannous fluoride, zinc citrate, zinc lactate, zinc phosphate, zinc oxide and combinations thereof.
1.7 The preceding oral care composition, wherein the composition comprises zinc citrate (e.g., in an amount from 0.2%-0.5% by wt.) (e.g., about 0.28% by wt.).
1.8 Any foregoing oral care composition wherein the quaternary ammonium surfactants is selected from cetylpyridinium chloride, chlorhexidine digluconate and mixtures thereof.
1.9 Any foregoing oral care composition wherein the quaternary ammonium surfactants is cetylpyridinium chloride (CPC), wherein the CPC is present in an amount from 0.005 to 0.05% by wt., wherein the wt % is relative to the total composition (e.g., from 0.0075%-0.025% by wt.,) (e.g., from 0.0075%-0.015% by wt.,), (e.g., about 0.01% by wt.), (e.g., about 0.015% by wt.), (e.g., about 0.02% by wt.).
1.10 Any foregoing oral care composition comprising an anionic surfactant.
1.11 The oral care composition of 1.10, wherein the anionic surfactant comprises an alkyl sulfate or an alkyl ether sulfate in free or orally acceptable salt form.
1.12 Any foregoing oral care composition wherein the anionic surfactant comprises a sodium, potassium, ammonium, and ethanolammonium salts of linear C8-C18 alkyl sulfate or C8-C18 alkyl ether sulfate.
1.13 Any foregoing oral care composition wherein the anionic surfactant comprises sodium laurel ether sulfate (SLES), sodium lauryl sulfate, and ammonium lauryl sulfate.
1.14 Any foregoing oral care composition wherein the anionic surfactant comprises sodium laurel sulfate.
1.15 Any foregoing oral care composition wherein the anionic surfactant is present in an amount sufficient to substantially interfere with interaction between a cationic active agent and the short chain polyphosphate salt, (e.g., an amount sufficient to inhibit formation of a precipitate or reduction of the efficacy of the cationic active agent).
1.16 Any foregoing oral care composition wherein the anionic surfactant is present in an amount of 0.01 to 5.0% by wt., 0.1 to 2.0% by wt., 0.1 to 1.0% by wt., 0.2 to 0.4% by wt., or about 0.33% by wt. of the total composition.
1.17 Any foregoing oral care composition further comprising a nonionic surfactant.
1.18 Any foregoing oral care composition comprising a nonionic surfactant selected from poloxamers or polyoxyethylene, e.g., poloxamer 407.
1.19 Any foregoing oral care composition comprising a nonionic surfactant which is a block copolymer of polyethylene glycol and polypropylene glycol,
1.20 Any foregoing oral care composition comprising a nonionic surfactant present in an amount from 0.01 to 5.0% by wt.; or 0.1 to 2.0% by wt.; or 0.1 to 0.6% by wt., 0.2 to 0.4% by wt.; or about 0.2% by wt.; or about 0.5%, all by wt. of the total composition.
1.21 Any foregoing oral care composition comprising a nonionic surfactant wherein the ratio (wt %) of anionic surfactant to nonionic surfactant is 5:1 to 1:5; 2:1 to 1:2; 1.5:1 to 1:1.5; 1.6:1; or 1:1.5.
1.22 Any foregoing oral care composition further comprising an amino acid or a polyamine, in free or orally acceptable salt form.
1.23 Any foregoing oral care composition comprising a polyamine, in free or orally acceptable salt form, selected from lysine or arginine, in free or orally acceptable salt form.
1.24 Any foregoing oral care composition wherein the composition comprises 0.01%-5% lysine, e.g., 0.5%-1.0% lysine, by wt. of the total composition, in free or orally acceptable salt form.
1.25 Any foregoing oral care composition wherein the composition comprises 0.01%-5% arginine by wt. of the total composition, in free or orally acceptable salt form, (e.g., 0.4%-2.0% arginine (e.g., e.g., 0.4%-1.5% arginine) (e.g., 1.5% arginine) (e.g., L-arginine) (e.g., arginine bicarbonate).
1.26 Any foregoing oral care composition wherein the composition comprises lysine in the form of a hydrochloride salt.
1.27 Any foregoing oral care composition wherein the composition comprises 0.01%-2.0% lysine hydrochloride, by wt. of the total composition.
1.28 Any foregoing oral care composition wherein the composition comprises 10%-90% by wt. of water wherein the weight is relative to the total composition (e.g., 15%-50% by wt.) (e.g., 15%-40% by wt.) (e.g., 15%-35% by wt.)
1.29 Any foregoing oral care composition wherein the composition comprises greater than 50% water, by wt. of the total composition (e.g., 50%-99% by wt.).
1.30 Any foregoing oral care composition wherein the composition comprises 70% to 95% water, by wt. of the total composition.
1.31 Any foregoing oral care composition wherein the composition comprises one or more of: a thickener, a buffer, a humectant, a surfactant, an abrasive, a sweetener, a flavorant, a pigment, a dye, an anti-caries agent, an anti-bacterial agent, a whitening agent, a desensitizing agent, a preservative, or a mixture thereof
1.32 Any foregoing oral care composition wherein the composition comprises a phosphate buffer.
1.33 Any foregoing oral care composition wherein the composition comprises a buffer wherein the buffer comprises sodium hydroxide.
1.34 Any foregoing oral care composition further comprising a pH adjustment agent selected from lactic acid, citric acid, hydrochloric acid, glycolic acid, sodium hydroxide, potassium chloride, monosodium citrate, disodium citrate, monosodium malate, sodium carbonate, bicarbonates, sesquicarbonates, borates, silicates; monosodium phosphate, trisodium phosphate, pyrophosphate salts, imidazole, or combinations thereof e.g., citric acid.
1.35 Any foregoing oral care composition comprising a pH adjustment agent in an amount of 0.01% to 5.0%, 0.01% to 2.0%, 0.1% to 1.0%, or about 0.5%, by wt. of the total composition.
1.36 Any foregoing oral care composition wherein the composition has a pH of about 1 to 7, about 3 to 6, about 5 to 6, or about 5.25 to 5.75, by wt. of the total composition.
1.37 Any foregoing oral care composition wherein the composition comprises a humectant, e.g., selected from: sorbitol, propylene glycol, glycerin, and combinations thereof
1.38 Any foregoing oral care composition wherein the composition a mixture of glycerin, sorbitol, and propylene glycol.
1.39 Any foregoing oral care composition, wherein the total humectant(s) weight comprises from 15%-30% by wt. (e.g., about 16% by wt.) (e.g., about 17% by wt.) (e.g., about 18% by wt.) of the total composition.
1.40 Any foregoing oral care composition wherein the composition comprises an abrasive.
1.41 Any foregoing oral care composition wherein the composition comprises an abrasive, wherein the abrasive comprises silica.
1.42 Any foregoing oral care composition wherein the composition comprises a sweetener.
1.43 Any foregoing oral care composition wherein the composition comprises a sweetener, and wherein the sweetener is sodium saccharin.
1.44 Any foregoing oral care composition wherein the composition comprises a flavorant.
1.45 Any foregoing oral care composition wherein the composition comprises a dye, e.g., FD&C Blue No. 1.
1.46 Any foregoing oral care composition wherein the composition comprises an anti-caries agent.
1.47 Any foregoing oral care composition wherein the composition comprises a fluoride ion source.
1.48 Any foregoing oral care composition wherein the composition comprises a fluoride ion source, wherein the fluoride ion source is stannous fluoride, sodium fluoride, potassium fluoride, sodium monofluorophosphate, sodium fluorosilicate, ammonium fluorosilicate, amine fluoride (e.g., N-octadecyltrimethylendiamine-N,N,N′-tris(2-ethanol)-dihydrofluoride), ammonium fluoride, titanium fluoride, hexafluorosulfate, or a mixture thereof.
1.49 Any foregoing oral care composition wherein the composition comprises a whitening agent.
1.50 Any foregoing oral care composition wherein the composition comprises a whitening agent, wherein the whitening agent is hydrogen peroxide.
1.51 Any foregoing oral care composition wherein the composition comprises a desensitizing agent, a vitamin, a preservative, an enzyme, or a mixture thereof.
1.52 Any foregoing oral care composition wherein the composition is a mouthwash, toothpaste, tooth gel, non-abrasive gel, mousse, foam, mouth spray, lozenge, oral tablet, dental implement, or pet care product.
1.53 Any foregoing oral care composition wherein the composition is a mouthwash.
1.54 Any foregoing oral care composition which is biphasic, e.g., wherein the solution comprises two distinct aqueous phases having different composition and density.
1.55 Any foregoing oral care composition which comprises less than 5%, e.g., less than 2% of hydrophobic ingredients.
1.56 Any foregoing oral care composition which is essentially oil-free, apart from flavoring agents.
1.57 Any foregoing oral care composition wherein there is no visible precipitation or reaction between the short chain polyphosphate salt and the orally acceptable cationic active agent after three months of storage at room temperature.
1.58 Any of the foregoing oral care compositions, wherein the preservative system comprises one or more polysorbates.
1.59 Composition of 1.58, wherein the polysorbate is selected from the group consisting of: polysorbate 20 (poly(ethylene oxide) (20) sorbitan monolaurate, Tween 20) or polysorbate 80 (poly(ethylene oxide) (80) sorbitan monolaurate, Tween 80)
1.60 Composition of 1.59, wherein the amount of the polysorbate (e.g., polysorbate 20) is from 1.25%-1.75%, by wt. of the total composition (e.g., from 1.25%-1.50%, by wt. of the total composition) (e.g., about 1.3% by wt.).
1.61 Any of the foregoing oral care compositions, wherein the amount of benzyl alcohol is between 0.05%-0.6% by wt. relative to the total weight of the composition (e.g., between 0.075%-0.2% by wt.) (e.g., between 0.085-0.15 by wt.) (e.g., about 0.1% by wt.) (e.g., about 0.2% by wt.) (e.g., about 0.3% by wt.) (e.g., about 0.4% by wt.) (e.g., about 0.5% by wt.)
1.62 The Composition of 1.61, wherein the amount of benzyl alcohol is from 0.075%-0.175%, by wt., relative to the total weight of the composition (e.g., 0.075%-0.125%, by wt.) (e.g., from 0.095%-0.115%, by wt.).
1.63 The Composition of 1.62, wherein the amount of benzyl alcohol is about 0.1% by wt., relative to the total weight of the composition.
1.64 The Composition of 1.62, wherein the amount of benzyl alcohol is about 0.15% by wt., relative to the total weight of the composition.
1.65 The Composition of 1.61, wherein the amount of benzyl alcohol is about 0.2% by wt., relative to the total weight of the composition.
1.66 Any foregoing oral care composition, wherein the oral care composition comprises:
   (i.) a preservative system, wherein the preservative system comprises:
      a. benzyl alcohol in an amount from 0.05%-0.2% by wt. (e.g., 0.075%-0.125% by wt.) (e.g., about 0.1%), (e.g., about 0.15% by wt.), (e.g., about 0.2% by wt.); and
      b. cetylpyridinium chloride (CPC)) in an amount from 0.005 to 0.025% by wt. (e.g., 0.005 to 0.02% by wt.), (e.g., about 0.01% by wt.), (e.g., about 0.015% by wt.), (e.g., about 0.02% by wt.);
      c. optionally a polysorbate; and
   (ii.) water.
   wherein all amounts are relative to the weight of the total composition.
1.67 Any foregoing oral care composition wherein the composition is a mouthwash, wherein the mouthwash comprises:
   (iii.) a preservative system, wherein the preservative system comprises:
      a. benzyl alcohol in an amount from 0.075%-0.125% by wt. (e.g., about 0.1% by wt.), (e.g., about 0.15% by wt.), (e.g., about 0.2% by wt.); and
      b. cetylpyridinium chloride (CPC)) in an amount from 0.005 to 0.02% by wt. (e.g., about 0.01% by wt.), (e.g., about 0.015% by wt.), (e.g., about 0.02% by wt.);
      c. optionally a polysorbate; and
   (iv.) water.
   wherein all amounts are relative to the weight of the total composition.
1.68 Any foregoing oral care composition wherein the composition is a mouthwash, wherein the mouthwash comprises:
   (i.) A preservative system, wherein the preservative system consists or consists essentially of:
      a. benzyl alcohol in an amount from 0.075%-0.125% by wt. (e.g., about 0.1%), (e.g., about 0.15% by wt.), (e.g., about 0.2% by wt.); and
      b. cetylpyridinium chloride (CPC)) in an amount from 0.005 to 0.02% by wt., (e.g., about 0.01% by wt.), (e.g., about 0.015% by wt.), (e.g., about 0.02% by wt.);
      c. optionally a polysorbate; and
   (ii.) water.
   wherein all amounts are relative to the weight of the total composition.
1.69 Any foregoing oral care composition wherein the composition is a mouthwash, wherein the mouthwash comprises:
   (i.) a preservative system, wherein the preservative system comprises:
      a. benzyl alcohol from 0.075%-0.125% by wt. (e.g., about 0.1% by wt.), (e.g., about 0.15% by wt.), (e.g., about 0.2% by wt.); and
      b. cetylpyridinium chloride (CPC)) from 0.005 to 0.02% by wt., (e.g., about 0.01% by wt.), (e.g., about 0.015% by wt.), (e.g., about 0.02% by wt.);
      c. optionally a polysorbate; and
   (ii.) zinc citrate
   (iii.) humectant mixture comprising: glycerin, sorbitol, and propylene glycol (e.g., 15%-20% by wt. of the humectant mixture relative to the weight of the composition) (e.g., from 15.5%-18.5% by wt.) (e.g., about 16% by wt.) (e.g., about 17% by wt.) (e.g., about 18% by wt.)); and
   (iv.) water;
   wherein all amounts are relative to the weight of the total composition.
1.70 Any of the forgoing oral care compositions comprising a short chain polyphosphate.
1.71 The composition of 1.70, wherein the short chain polyphosphate is selected from the group consisting of: alkali dibasic phosphate and alkali pyrophosphate salts, e.g., sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these.
1.72 The composition of 1.71, wherein the short chain polyphosphate is selected from the group consisting of: tetrasodium pyrophosphate (TSPP), sodium tripolyphosphate (STPP), tetrapotassium pyrophosphate (TKPP), and mixtures thereof.
1.73 The composition of 1.72, wherein the short chain polyphosphate is tetrasodium pyrophosphate.
1.74 Any of the forgoing oral care compositions comprising a thickener or gum.
1.75 The oral care composition of 1.74, wherein the thickener or gum is xanthan gum.
1.76 Any of the forgoing oral care compositions, wherein preservative system does not affect the overall flavor of the aqueous oral care composition.
1.77 Any of the preceding oral care compositions, wherein the further polymerizable ethylenically unsaturated monomer comprises methyl vinyl ether (methoxyethylene).
1.78 Any of the preceding oral care compositions, wherein the PVM/MA copolymer comprises a copolymer of methyl vinyl ether/maleic anhydride, wherein the anhydride is hydrolyzed following copolymerization to provide the corresponding acid.
1.79 Any of the preceding oral care compositions, wherein the PVM/MA copolymer comprises a GANTREZ® polymer (e.g., GANTREZ® S-97 polymer)
1.80 Any of the preceding oral care compositions wherein the pH is between 7.5 and 10.5. e.g., about 7.5 or about 8.0.
1.81 Any of the preceding oral care oral care compositions, wherein the oral care composition comprises:
   (i) a preservative system, wherein the preservative system comprises:
      a. benzyl alcohol at about 0.1% by wt., relative to the total composition; and
      b. cetylpyridinium chloride (CPC)) at about 0.01% by wt., relative to the total composition; and
   (ii) Zinc citrate;
   (iii) humectant mixture comprising: glycerin, sorbitol, and propylene glycol (e.g., 15%-20% by wt. of the humectant mixture relative to the weight of the composition (e.g., about 16% by wt.) (e.g., about 17% by wt.) (e.g., about 18% by wt.)); and
   (iv) water;
   wherein all amounts are by weight relative to the total composition.
1.82 Any preceding oral care composition, wherein the oral care composition is in the form selected from: dentifrice (e.g., a toothpaste or oral gel), cream, mouthwash, strip or gum (e.g., chewing gum).
1.83 The preceding oral care composition wherein the oral care composition is a mouthwash.
1.84 Any foregoing oral care composition, wherein the oral care composition comprises a preservative system and wherein the preservative system comprises benzyl alcohol and cetylpyridinium chloride (CPC)) in a ratio (wt %) of benzyl alcohol to CPC of: 15:1 to 1:1 (benzyl alcohol:CPC); or 12:1 to 5:1 (benzyl alcohol:CPC); or 11:1 to 7:1 (benzyl alcohol:CPC); about 10:1 (benzyl alcohol:CPC); or 10:1 (benzyl alcohol:CPC).

In a further aspect the compositions of the disclosure contemplate the use of an anionic surfactant, in free or orally acceptable salt form, to stabilize an oral care formulation comprising a short chain polyphosphate salt and an effective amount of orally acceptable cationic active agent, in free or orally acceptable salt form; for example, the use being in any of the foregoing Composition 1.0, et seq.

As used herein, an “oral care composition” refers to a composition for which the intended use can include oral care, oral hygiene, or oral appearance, or for which the intended method of use can comprise administration to the oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrices and mouthwashes. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

As used herein, “short chain polyphosphate salt” encompasses orally acceptable mono- and polyphosphates, for example, P₁₋₆ phosphates such as monobasic, dibasic or tribasic phosphate; and dimeric phosphates, e.g., sodium hexametaphosphate. For example, the short chain polyphosphate salt may comprise alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetra pyrophosphate (Na₄P₂O₇), calcium pyrophosphate (Ca₂P₂O₇), and sodium phosphate dibasic (Na₂HPO₄). In one embodiment, tetrasodium pyrophosphate (TSPP), sodium tripolyphosphate (STPP), tetrapotassium pyrophosphate (TKPP), or mixtures thereof are used. In another embodiment, the compositions comprise a mixture of tetrapotassium pyrophosphate (TSPP) and sodium tripolyphosphate (STPP)(Na₅P₃O₁₀). In one aspect, the aforementioned phosphates are provided in an amount effective to reduce stains on tooth surfaces, erosion of the enamel, to aid in cleaning the teeth, and/or reduce tartar buildup on the teeth, for example, in any of Composition 1.0 et. seq, in an amount of 0.01 wt. % to 5.0 wt. %, 0.1 wt. % to 5.0 wt. %, 0.1 wt. % to 3 wt. %, 0.5 wt. % to 1.5 wt. %, or 1.0 wt. % based on the total weight of the composition.

As used herein, “orally acceptable cationic active agent” means an agent which is cationic in aqueous solution at neutral pH and which provides some benefit, e.g. antimicrobial, antigingivitis, and/or antierosion activity, to the teeth or oral cavity. While in aqueous formulation, the agent will generally be in solution, but it may be introduced to the formulation formulated in free or orally acceptable salt form. In certain embodiments, the orally acceptable cationic active agent is selected from one or more of quaternary ammonium surfactants (such as cetylpyridinium chloride (CPC)), bisguanides (such as chlorhexidine digluconate), cationic amino acids (such as arginine), metal cations (such as zinc, calcium, or stannous ions), or combinations thereof. In one aspect, any of Compositions 1.0, et seq can comprise an orally acceptable cationic active agent.

As used herein, “anionic surfactant” means those surface-active or detergent compounds that contain an organic hydrophobic group containing generally 8 to 26 carbon atoms or generally 10 to 18 carbon atoms in their molecular structure and at least one water-solubilizing group selected from sulfonate, sulfate, and carboxylate so as to form a water-soluble detergent. Usually, the hydrophobic group will comprise a C₈-C₂₂ alkyl, or acyl group. Such surfactants are employed in the form of water-soluble salts and the salt-forming cation usually is selected from sodium, potassium, ammonium, magnesium and mono-, di- or tri-C₂-C₃ alkanolammonium, with the sodium, magnesium and ammonium cations again being the usual ones chosen. Some examples of suitable anionic surfactants include, but are not limited to, the sodium, potassium, ammonium, and ethanolammonium salts of linear C₈-C₁₈ alkyl ether sulfates, ether sulfates, and salts thereof. Suitable anionic ether sulfates have the formula R(OC₂H₄)ₙOSO₃M wherein n is 1 to 12, or 1 to 5, and R is an alkyl, alkylaryl, acyl, or alkenyl group having 8 to 18 carbon atoms, for example, an alkyl group of C₁₂-C₁₄ or C₁₂-C₁₆, and M is a solubilizing cation selected from sodium, potassium, ammonium, magnesium and mono-, di- and triethanol ammonium ions. Exemplary alkyl ether sulfates contain 12 to 15 carbon atoms in the alkyl groups thereof, e.g., sodium laureth (2 EO) sulfate. Some preferred exemplary anionic surfactants that may be used in the compositions of the present disclosure include sodium laurel ether sulfate (SLES), sodium lauryl sulfate, and ammonium lauryl sulfate. In certain embodiments, the anionic surfactant is present in an amount of 0.01 to 5.0%, 0.1 to 2.0%, 0.2 to 0.4%, or about 0.33% by wt. In one aspect, any of Compositions 1.0, et seq can comprise an anionic surfactant as described herein.

As used herein, “nonionic surfactant” generally refers to compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, alkyl polyglycosides (for example, fatty alcohol ethers of polyglycosides, such as fatty alcohol ethers of polyglucosides, e.g., decyl, lauryl, capryl, caprylyl, myristyl, stearyl and other ethers of glucose and polyglucoside polymers, including mixed ethers such as capryl/caprylyl (C₈₋₁₀) glucoside, coco (C₈₋₁₆) glucoside, and lauryl (C₁₂₋₁₆) glucoside), long chain tertiary amine oxides, long chain tertiary phosphine oxides, long chain dialkyl sulfoxides, and mixtures of such materials. In one aspect, any of Compositions 1.0, et seq can comprise a nonionic surfactant.

In some embodiments, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and mixtures thereof. Examples of fatty acid amides include, but are not limited to, cocomonoethanolamide, lauramide monoethanolamide, cocodiethanolamide, and mixtures thereof. In certain embodiments, the nonionic surfactant is a combination of an amine oxide and a fatty acid amide. In certain embodiments, the amine oxide is a mixture of laurylamidopropyl dimethylamine oxide and myristylamidopropyl dimethylamine oxide. In certain embodiments, the nonionic surfactant is a combination of lauryl/myristylamidopropyl dimethylamine oxide and cocomonoethanolamide. In certain embodiments (e.g., any of Compositions 1.0, et seq), the nonionic surfactant is present in an amount of 0.01 to 5.0%, 0.1 to 2.0%, 0.1 to 0.6%, 0.2 to 0.4%, about 0.2%, or about 0.5% by wt.

As used herein “polyamine compound” means a molecule having at least two primary or secondary amine groups, for example having an isoelectric point of greater than pH 8.5, for example pH 9-10. Examples of polyamines include ethylene diamine, lysine, or histadine, as well as polymers such as Lupasol P, which is a polyethylenimine. The polyamine must be safe for its intended use. Where the composition is an oral care composition, the polyamine must be orally acceptable. The polyamine may be provided in free or acid addition salt form. In certain embodiments the polyamine compound is lysine. In one aspect, any of Compositions 1.0, et seq can comprise a polyamine compound.

As used herein, “biphasic” refers to stable liquid compositions which contain at least two distinct homogeneous phases, having different densities, such that the phases are separate at rest. The phases may be readily mixed by shaking but will then re-separate over a short period, e.g., less than half an hour. In certain embodiments, the term excludes gels, emulsions, microemulsions, and homogeneous solutions. In certain embodiments, these formulations differ from conventional biphasic formulations in that both phases are aqueous, rather than one phase being hydrophobic and the other hydrophilic.

In certain aspects, any of Compositions 1.0 et seq can comprise a basic or neutral amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine. In certain embodiments, the basic amino acid is arginine, for example, L-arginine, or a salt thereof.

In another aspect, the compositions of the invention (e.g., Compositions 1.0 et seq) can include a neutral amino acid, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions of the disclosure (e.g., any of Composition 1.0 et seq) are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

As used herein, a “tartar control agent” refers to a compound or a mixture of compounds that inhibit the formation of tartar, a mixture of calcium phosphates on organic matrices, and/or the deposition of plaque on teeth to form tartar (calculus).

As used herein, “chemical stain” refers to a discoloration of a dental surface caused by adsorption or absorption of a colored agent on or into the surface, or caused by chemical reaction of material of the dental surface (e.g., dental enamel) with a colored or noncolored agent contacting the surface. “Chemical staining” herein means formation and/or development of a chemical stain.

As used herein, “dental surface” refers to a surface of a natural tooth or a hard surface of artificial dentition including a crown, cap, filling, bridge, dental implant and the like. In some embodiments, the dental surface is a natural tooth.

As used herein, the term “polysorbate” means oleate esters of sorbitol and its anhydrides, typically copolymerized with ethylene oxide.

The compositions are, for example, oral care compositions, in accordance with any of Compositions 1.0, et seq. for example mouthwashes. Any of the compositions of Compositions 1.0, et seq. are suitable for oral care use provided the ingredients are orally acceptable. In some embodiments, the mouthwash of Composition 1.0, et seq comprises an effective amount of an orally acceptable cationic active agent, which is an antimicrobial, antigingivitis, anti-erosion and/or anti-caries agent, e.g. a cationic active agent selected from one or more of quaternary ammonium surfactants (such as cetylpyridinium chloride (CPC)), bisguanides (such as chlorhexidine digluconate), cationic amino acids (such as arginine), metal cations (such as zinc, calcium, or stannous ions), or combinations thereof. The orally acceptable cationic active agent may be present in an effective amount, for example an antimicrobial, antigingivitis, anti-erosion and/or anti-caries amount. The precise amount will depend on the particular active agent and the condition to be treated or prevented, but in various embodiments, antimicrobially effective levels of CPC in a mouthwash would include amounts from 0.005 to 0.05%, e.g., about 0.01% by wt.

The oral care composition used in the present disclosure comprise significant levels of water. Water employed in the preparation of commercial oral compositions should be deionized and free of organic impurities. The amount of water in the compositions includes the free water that is added plus that amount which is introduced with other materials.

Mouthwashes frequently contain significant levels of ethanol, which is often needed to solubilize essential oils and to prevent bacterial contamination. High levels of ethanol may be undesirable, because in addition to the potential for abuse by ingestion, the ethanol may exacerbate conditions like xerostoma. Accordingly, in some embodiments, the oral care compositions of the disclosure (e.g., any of Composition 1.0, et seq.) are substantially free of ethanol, e.g., contain less than 1% ethanol.

In some aspect, the oral care compositions of the disclosure (e.g., any of Composition 1.0, et seq.) can comprise one or more humectants. Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case. In some embodiments, the oral care compositions of the disclosure (e.g., any of Composition 1.0, et seq.), comprise one or more humectants present at levels of 5% to 30%, e.g., 10% to 20% by weight.

Suitable thickeners include naturally occurring polymers such as carrageenan, xanthan gum, polyglycols of various molecular weights sold under the trade name Polyox, and polyvinylpyrrolidone.

Flavorings for use in the compositions of the disclosure may include extracts or oils from flavorful plants such as peppermint, spearmint, cinnamon, wintergreen, and combinations thereof, cooling agents such as menthol, methyl salicylate, and commercially available products such as OptaCool® from Symrise, as well as sweeteners, which may include polyols (which also function as humectants), saccharin, acesulfame, aspartame, neotame, stevia and sucralose.

Further provided is a method (Method A) for the treatment and/or inhibition of a chemical stain, plaque, and/or tartar on a dental surface, comprising contacting the dental surface with any of the preceding oral care compositions.

Further provided herein is Method A as follows:
A.1 Method A wherein the composition is any of Composition 1.0 et seq, e.g., selected from any of Compositions 1.0-1.84.
A.2 Method A or A.1 wherein the method is for the treatment of a chemical stain, plaque, and/or tartar on the dental surface.
A.3 Method A.2 wherein the method is for the treatment of a chemical stain on the dental surface.
A.4 Method A.2 wherein the method is for the treatment of plaque on the dental surface.
A.5 Method A.2 wherein the method is for the treatment of tartar on the dental surface.
A.6 Method A or A.1 wherein the method is for the inhibition of a chemical stain, plaque, and/or tartar on the dental surface.
A.7 Method A.6 wherein the method is for the inhibition of a chemical stain on the dental surface.
A.8 Method A.6 wherein the method is for the inhibition of plaque on the dental surface.
A.9 Method A.6 wherein the method is for the inhibition of tartar on the dental surface.
A.10 Method A or A.1-A.9 wherein the dental surface is a human tooth.
A.11 Method A or A.1-A.10 wherein the composition is contacted with the dental surface by brushing.
A.12 Any foregoing Method A, el seq. wherein the formulation is biphasic and is shaken before use.

Further provided is a method (Method B) for the treatment and/or inhibition of gum disease comprising contacting the oral cavity with any of the preceding oral care compositions.

Further provided herein is Method B as follows:
B.1 Method B wherein the composition is any of Compositions 1.0 et seq, e.g., any of Compositions 1.0-1.84.
B.2 Method B or B.1 wherein the method is for the treatment of gum disease.
B.3 Method B, B.1, or B.2 wherein the gum disease is gingivitis.
B.4 Method B, B.1, or B2 wherein the gum disease is periodontitis.
B.5 Method B or B.1 wherein the method is for the inhibition of gum disease.
B.6 Method B, B.1, or B.5 wherein the gum disease is gingivitis.
B.7 Method B, B.1, or B.5 wherein the gum disease is periodontitis.
B.8 Method B or B.1-B.7 wherein the oral cavity is a human oral cavity.
B.9 Method B or B.1-B.8 wherein the composition is contacted with the oral cavity by brushing.
B.10 Any foregoing Method B, wherein the formulation is biphasic and is shaken before use.

Further provided is a method (Method C) for the treatment and/or inhibition of halitosis comprising contacting the oral cavity with any of the preceding oral care compositions.

Further provided herein is Method C as follows:
C.1 Method C wherein the composition is any of Compositions 1.0 et seq., e.g., any of Compositions 1.0-1.84.
C.2 Method C or C.1 wherein the oral cavity is a human oral cavity.
C.3 Method C, C.1, or C.2 wherein the composition is contacted with the oral cavity by brushing.
C.4 Any foregoing Method C, et seq. wherein the formulation is biphasic and is shaken before use.

Further provided is a method (Method D) for inhibiting biofilm formation on a dental surface comprising contacting the dental surface with any of the preceding oral care compositions.

Further provided herein is Method D as follows:
D.1 Method D wherein the composition is any of Compositions 1.0 et seq., e.g., any of Compositions 1.0-1.84.
D.2 Method D or D.1 wherein the dental surface is a human tooth.
D.3 Method D, D.1, or D.2 wherein the composition is contacted with the dental surface by brushing.
D.4 Any foregoing Method D, el seq. wherein the formulation is biphasic and is shaken before use.

Further provided is a method (Method E) for treating and/or inhibiting bacteria from aggregating and forming bigger colonies in an oral cavity comprising contacting the oral cavity with any of the preceding oral care compositions.

Further provided herein is Method E as follows:
E.1 Method E wherein the composition is any of Compositions 1.0 et seq., e.g., any of Compositions 1.0-1.84.
E.2 Method E or E.1 wherein the oral cavity is a human oral cavity.
E.3 Method E, E.1, or E.2 wherein the composition is contacted with the oral cavity by brushing.
E.4 Any foregoing Method E, et seq. wherein the formulation is biphasic and is shaken before use.

Further provided are any of Compositions 1.0, et seq. for use in any of Methods A-E.

As used herein, “inhibition” refers to reduction of stains that would otherwise form or develop subsequent to the time of the treatment. Such inhibition can range from a small but observable or measurable reduction to complete inhibition of subsequent staining, by comparison with an untreated or placebo-treated dental surface.

Where the dental surface is substantially free of chemical stains, Method A, e.g., A.1-A.12, is effective to inhibit formation and development of new chemical stains, as can occur for example by oral use of tobacco products (including smoking) or by drinking tea, coffee, red wine, or coke, subsequent to treatment according to the method. Where the dental surface already possesses some degree of chemical staining, Method A, e.g., A.1-A.12, is effective to inhibit further development of the existing stain. In some embodiments, the Method A, e.g., A.1-A.12, can remove, partially or completely, an existing chemical stain as well as inhibit subsequent staining.

In another embodiment, the disclosure provides an oral care composition or oral composition premix, comprising a preservative system, wherein the preservative system comprises: benzyl alcohol from 0.075%-0.125% by wt. (e.g., about 0.1%, about 0.15%, or about 0.2% by wt.); and cetylpyridinium chloride (CPC)) from 0.005 to 0.02% by wt., e.g., about 0.01% by wt., e.g., about 0.015% by wt., e.g., about 0.02% by wt.; optionally a polysorbate; and water (e.g., an oral care composition according to any of Compositions 1, et seq.) obtained or obtainable by the process of Method F.

### EXAMPLES

### Example 1

The preservative system of the present disclosure is tested in various commercial mouthwash formulations to determine how micro-robustness and flavor may be affected. Samples are tested over the course of weeks in an ageing study.

Preservative systems having combinations with lower amounts of benzyl alcohol, e.g., about 0.1% by wt. Benzyl Alcohol, and CPC, e.g., about 0.01% by wt., are believed to provide adequate micro-robustness—for example, compared to commercial preservative systems and existing industry standards—without adversely affecting flavor. Moreover, this combination is believed to allow for the use of less benzyl alcohol than some commercial preservative combinations that employ CPC and polysorbate.

Table 1 demonstrates APET with formulas with varying concentrations of benzyl alcohol:

| | | | | APET | APET Aged |
|---|---|---|---|---|---|
| Formula | Flavor | Flavor | APET B. | Aged | Log Red Mold |
| (by wt. %) * | Initial | Aged | Stabilis | Bacteria | 21/28 days |
| 0.15% Benzyl | Pass | Pass | Pass | Pass | 1.1/1.7 |
| Alcohol/1.43% | | | | | |
| Polysorbate | | | | | |
| 0.2% Benzyl | Pass | Pass | Pass | Pass | 1.1/2.7 |
| Alcohol/1.43% | | | | | |
| Polysorbate | | | | | |
| 0.1% Benzyl | Pass | Pass | Pass | Pass | 2.2/4.0 |
| Alcohol/0.01% | | | | | |
| CPC | | | | | |

(* % by wt. amounts are relative to the total wt. % of the sample)

The Antimicrobial Preservative Effectiveness Test (APET) is a 28 days test that includes inoculating two product samples with separate pools: a bacteria pool and a mold pool. Subsequently, the samples are homogenized and incubated for seven days. After incubation, an aliquot is taken and the amount of the mold and microorganisms/bacteria that survive are counted. Next, a new bacteria pool is added into the sample and incubated for another seven days. After the seven days (14 days total), another aliquot is taken and the amount of the microorganisms/bacteria that survive is counted. This procedure is repeated for a total of 28 days.

As demonstrated by Table 1, samples with 0.1% by wt. of benzyl alcohol and 0.01% by wt. of CPC demonstrate acceptable bacterial efficacy. Furthermore, lower amounts of benzyl alcohol (0.1% by wt.) when coupled with CPC (0.01% by wt.) demonstrate increased efficacy against mold compared to samples with higher amounts of benzyl alcohol and polysorbate without any CPC. Note the flavor is acceptable in all samples listed in Table 1.

### Example 2

The following is a representative mouthwash formula of the oral care compositions described herein:

TABLE A

| Ingredients | Amount (by wt. %) |
|---|---|
| DEMINERALIZED WATER | q.s. |
| | (e.g., 70%-80% |
| | by wt.) |
| GLYCERIN - USP, EP VEG | 7.5 |
| SORBITOL - 70% SOLUTION | 5.5 |
| PROPYLENE GLYCOL USP, | 5.0 |
| EP | |
| POLYSORBATE 20 USP, EP | 1.3 |
| ZINC CITRATE | 0.28 |
| TRIHYDRATE | |
| COLOR, SWEETENER, | 0.20 |
| FLAVOR | |
| SODIUM FLUORIDE - USP, | 0.05 |
| EP | |
| ALKALI PYROPHOSPHATE | 1.8 |
| SALT | |
| BENZYL ALCOHOL FCC | 0.1 |
| CETYLPYRIDINIUM | 0.01 |
| CHLORIDE USP | |
| PVM/MA COPOLYMER | 1.5 |
| Total Components | 100.0 |

1. An aqueous oral care composition comprising:
(i.) A preservative system, wherein the preservative system comprises:
a. an effective amount of benzyl alcohol, wherein the amount of benzyl alcohol is present in an amount from 0.05%-0.2% by wt. of the total composition; and
b. an effective amount of cetylpyridinium chloride (“CPC”), wherein the amount CPC is present in an amount from 0.005 to 0.025% by wt. of the total composition;
c. optionally an effective amount of a polysorbate; and
(ii.) water.

2. The oral care composition of claim 1, further comprising an anionic surfactant.

3. The oral care composition of claim 1, further comprising a nonionic surfactant.

4. The oral care composition of claim 1, wherein the composition comprises greater than 50% water, by wt. of the total composition.

5. The oral care composition of claim 1, wherein the composition comprises a flavorant.

6. The oral care composition of claim 1, wherein the composition comprises an anti-caries agent.

7. The oral care composition of claim 1, wherein the composition comprises a fluoride ion source.

8. The oral care composition of claim 1, wherein the composition comprises a whitening agent.

9. The oral care composition of claim 1, wherein the composition is a mouthwash.

10. The oral care composition of claim 1, wherein the amount of benzyl alcohol is from 0.075%-0.125% by wt., wherein the wt % is relative to the total weight of the composition.

11. The oral care composition of any of claim 1, wherein the CPC is present in an amount of 0.005% to 0.02% by wt. %, wherein the wt % is relative to the total weight of the composition.

12. The oral care composition of any of claim 1, wherein the composition is a mouthwash, wherein comprising:
(i.) a preservative system, wherein the preservative system comprises:
a. benzyl alcohol from 0.075%-0.125% by wt.; and
b. cetylpyridinium chloride (CPC)) from 0.005 to 0.02% by wt.;
c. optionally a polysorbate; and
(ii.) water;
wherein all amounts are by weight of the total composition.

13. The oral care composition of claim 1, wherein the oral care composition is in the form selected from: dentifrice, cream, mouthwash, strip or gum.

14. A method for:
a) the treatment and/or inhibition of a chemical stain, plaque, and/or tartar on a dental surface,
b) the treatment and/or inhibition of gum disease,
c) the treatment and/or inhibition of halitosis,
d) inhibiting biofilm formation on a dental surface, and/or
e) treating and/or inhibiting bacteria from aggregating and forming bigger colonies in an oral cavity
comprising contacting a dental surface with an oral care composition according to claim 1.
